(19) United States
(12) Patent Application Publication
(10) Pub. No.: US 2026/0283784 A1
(43) Pub. Date: Sep. 24, 2026

(54) INTRAOCULAR PHAKIC IMPLANT AND METHODS OF CORRECTING FOR PRESBYOPIA WITH AN INTRAOCULAR PHAKIC IMPLANT

(71) Applicant: Alcon Inc., Fribourg (CH)

(72) Inventor: Terah Whiting SMILEY, Davis, CA (US)

(73) Assignee: Alcon Inc., Fribourg (CH)

(21) Appl. No.: 19/449,307

(22) Filed: Jan. 14, 2026

Related U.S. Application Data
(60) Provisional application No. 63/775,574, filed on Mar. 21, 2025.

(51) Int. Cl. A61F 2/16; A61F 2/00

(52) U.S. Cl. CPC A61F 2/1601; A61F 2002/0081; A61F 2002/1683; A61F 2002/1689; A61F 2230/0006; A61F 2230/0013; A61F 2250/0018; A61F 2250/0053

(57) ABSTRACT

Disclosed are intraocular phakic implants and methods of correcting for presbyopia with an intraocular phakic implant. In one aspect, an intraocular phakic implant comprises an opaque portion comprising a pinhole opening defined along the opaque portion and a plurality of haptics coupled to or extending from the opaque portion. Also disclosed is a method of increasing a depth of focus of an eye with an intraocular phakic implant.

### CROSS-REFERENCE TO RELATED APPLICATION

This application claims the benefit of priority to U.S. Provisional Patent Application No. 63/775,574 filed on Mar. 21, 2025, the content of which is incorporated herein by reference in its entirety.

### TECHNICAL FIELD

The present disclosure relates generally to the field of intraocular implants, and, more specifically, to intraocular phakic implants and methods of correcting for presbyopia with an intraocular phakic implant.

### BACKGROUND

Presbyopia is a gradual loss of accommodation of the eye. As a person ages, the crystalline lens of the eye becomes less flexible and elastic. Moreover, muscles in the eye called ciliary muscles, which are responsible for pushing and pulling the crystalline lens, also become less effective as a person ages. These changes result in the crystalline lens being unable to adequately adjust, which causes objects that are close to the eye to appear blurry.

In most individuals, the symptoms of presbyopia begin to become noticeable under normal viewing conditions after age 40. Eventually, the ability of the eye to undergo accommodation is reduced to the point at which the individual is unable to focus on objects close at hand. Individuals that are presbyopic typically have difficulty reading small print, including on computer display monitors and smartphones.

Current ways to manage presbyopia or for those undergoing this gradual loss of accommodation include bifocal or reading glasses, presbyopia eye drops, and bifocal or monovision contact lenses. However, contact lenses are not well tolerated by some individuals. In addition, presbyopia eye drops must be administered regularly and can lead to red eyes and headaches. Furthermore, many individuals suffering from presbyopia would rather not wear bifocal glasses or reading glasses. This is especially true for individuals that are just starting to notice signs of presbyopia.

Therefore, a solution is needed which can correct for presbyopia without requiring an individual to take regular eye drops, wear contact lenses, or wear unsightly glasses. Moreover, such a solution should not be overly complicated and should suit an individual's optic needs prior to undergoing pseudophakic cataract surgery.

### SUMMARY

Disclosed herein are intraocular phakic implants and methods of correcting for presbyopia with an intraocular phakic implant. In some embodiments, an intraocular phakic implant is disclosed comprising an opaque portion and a plurality of haptics coupled to or extending from the opaque portion.

The opaque portion can comprise a pinhole opening defined along the opaque portion. In some embodiments, the opaque portion is devoid of any refractive power or dioptric power.

In some embodiments, each of the haptics can be an arm-type haptic shaped as a partial spiral. In other embodiments, each of the haptics can be a plate-type haptic or wing-type haptic.

In some embodiments, each of the haptics can be configured to bend or curl in an anterior direction to allow the haptics to engage with the ciliary sulcus to fixate the intraocular phakic implant within an eye.

In some embodiments, a stiffness or rigidity of the haptics can be greater than the stiffness or rigidity of the opaque portion.

In some embodiments, the opaque portion can have a lubricious or slippery surface texture.

In some embodiments, the opaque portion can be shaped substantially as a flattened dome.

In some embodiments, the opaque portion can have a thickness of between about 0.1 mm and about 0.5 mm.

In some embodiments, the pinhole opening can have an opening diameter of between about 1.0 mm and about 3.0 mm. In certain embodiments, the pinhole opening can have an opening diameter of between about 1.5 mm and about 2.0 mm (e.g., about 1.8 mm).

In some embodiments, the opaque portion can have a portion outer diameter of between about 6.0 mm and about 8.0 mm. In certain embodiments, the opaque portion can have a portion outer diameter of between about 6.5 mm and about 7.5 mm.

In some embodiments, the pinhole opening can be substantially circular. In other embodiments, the pinhole opening can be substantially elliptical or oval.

In some embodiments, the pinhole opening can be configured to allow aqueous humor and other fluids to flow through the pinhole opening.

In some embodiments, the opaque portion can be made of a biocompatible polymeric material. The opaque portion can be formed by directing laser light at the biocompatible polymeric material until the biocompatible polymeric material carbonizes and turns opaque or black.

In some embodiments, the opaque portion can comprise a black-colored pigment. For example, the opaque portion can comprise the pigment carbon black.

In some embodiments, the opaque portion and the haptics can have rounded edges and ends.

Also disclosed is a method of correcting for presbyopia. The method can comprise forming an incision in a cornea of an eye and introducing an intraocular phakic implant into a posterior chamber of the eye until the intraocular phakic implant is implanted or positioned between the crystalline lens of the eye and the iris of the eye.

In some embodiments, the eye can be a non-dominant eye.

In some embodiments, the intraocular phakic implant can comprise an opaque portion having a pinhole opening defined along the opaque portion.

In some embodiments, the pinhole opening can be configured to allow aqueous humor and other fluids within the eye to flow through the pinhole opening.

In some embodiments, the pinhole opening can have an opening diameter. The opening diameter can be between about 1.0 mm and about 3.0 mm. In certain embodiments, the pinhole opening can have an opening diameter of between about 1.5 mm and about 2.0 mm (e.g., about 1.8 mm).

In some embodiments, the opaque portion can have a portion outer diameter between about 6.0 mm and about 8.0 mm. In certain embodiments, the opaque portion can have a portion outer diameter of between about 6.5 mm and about 7.5 mm.

In some embodiments, the opaque portion can be devoid of any refractive power or dioptric power.

In some embodiments, the opaque portion can have a lubricious or slippery surface texture.

In some embodiments, the opaque portion can be shaped substantially as a flattened dome.

In some embodiments, the opaque portion can have a thickness of between about 0.1 mm and about 0.5 mm.

In some embodiments, the opaque portion can be made of a biocompatible polymeric material. The opaque portion can be formed by directing laser light at the biocompatible polymeric material until the biocompatible polymeric material carbonizes and turns opaque or black.

In some embodiments, the opaque portion can comprise a black-colored pigment. For example, the opaque portion can comprise the pigment carbon black.

In some embodiments, the intraocular phakic implant can comprise a plurality of haptics configured to engage the ciliary sulcus of the eye to fixate the intraocular phakic implant within the eye.

In some embodiments, the haptics can comprise rounded edges and ends. Each of the haptics can be configured to bend or curl in an anterior direction to allow the haptics to engage with the ciliary sulcus to fixate the intraocular phakic implant within the eye.

In some embodiments, each of the haptics can be an arm-type haptic shaped as a partial spiral. In other embodiments, each of the haptics can be a plate-type haptic or wing-type haptic.

In some embodiments, the method can further comprise rolling up the intraocular phakic implant and introducing the intraocular phakic implant into the eye when the intraocular phakic implant is rolled-up.

In some embodiments, the method can further comprise removing the intraocular phakic implant from the eye prior to cataract surgery.

Further disclosed is a method of increasing a depth of focus of an eye. The method can comprise forming an incision in a cornea of the eye and introducing an intraocular phakic implant into a posterior chamber of the eye. The intraocular phakic implant can comprise an opaque portion having a pinhole opening defined along the opaque portion and a plurality of haptics coupled to or extending from the opaque portion.

In some embodiments, the pinhole opening can be configured to allow aqueous humor and other fluids within the eye to flow through the pinhole opening.

In some embodiments, the pinhole opening can have an opening diameter between about 1.0 mm and about 3.0 mm. In certain embodiments, the opening diameter can be between about 1.5 mm and about 2.0 mm (e.g., about 1.8 mm).

In some embodiments, the opaque portion can have a portion outer diameter between about 6.0 mm and about 8.0 mm. In certain embodiments, the portion outer diameter of between about 6.5 mm and about 7.5 mm.

In some embodiments, the opaque portion is devoid of any refractive power or dioptric power.

In some embodiments, the opaque portion can have a lubricious or slippery surface texture.

In some embodiments, the opaque portion can be shaped substantially as a flattened dome.

In some embodiments, the opaque portion can have a thickness of between about 0.1 mm and about 0.5 mm.

In some embodiments, the opaque portion can be made of a biocompatible polymeric material. The opaque portion can be formed by directing laser light at the biocompatible polymeric material until the biocompatible polymeric material carbonizes and turns opaque or black.

In some embodiments, the opaque portion can comprise a black-colored pigment. For example, the opaque portion can comprise the pigment carbon black.

In some embodiments, the haptics can be configured to engage the ciliary sulcus of the eye to fixate the intraocular phakic implant within the eye. The haptics can have rounded edges and ends.

In some embodiments, each of the haptics can be configured to bend or curl in an anterior direction to allow the haptics to engage with the ciliary sulcus to fixate the intraocular phakic implant within the eye.

In some embodiments, each of the haptics can be an arm-type haptic shaped as a partial spiral. In other embodiments, each of the haptics can be a plate-type haptic or wing-type haptic.

In some embodiments, the method can further comprise rolling up the intraocular phakic implant and introducing the intraocular phakic implant into the eye when the intraocular phakic implant is rolled-up. In certain embodiments, the intraocular phakic implant can be introduced into the eye of a subject.

### BRIEF DESCRIPTION OF THE DRAWINGS

FIG. 1 illustrates a perspective view of one embodiment of an intraocular phakic implant.

FIG. 2 illustrates a top plan view of the intraocular phakic implant of FIG. 1.

FIG. 3 illustrates a side cross-sectional view of part of the intraocular phakic implant.

FIG. 4 illustrates a perspective view of another embodiment of an intraocular phakic implant.

FIG. 5 is a schematic diagram illustrating a side cross-sectional view of the intraocular phakic implant implanted within the eye.

FIG. 6 illustrates one embodiment of a method of correcting for presbyopia with an intraocular phakic implant.

FIG. 7 illustrates one embodiment of a method for increasing a depth of focus of an eye.

### DETAILED DESCRIPTION

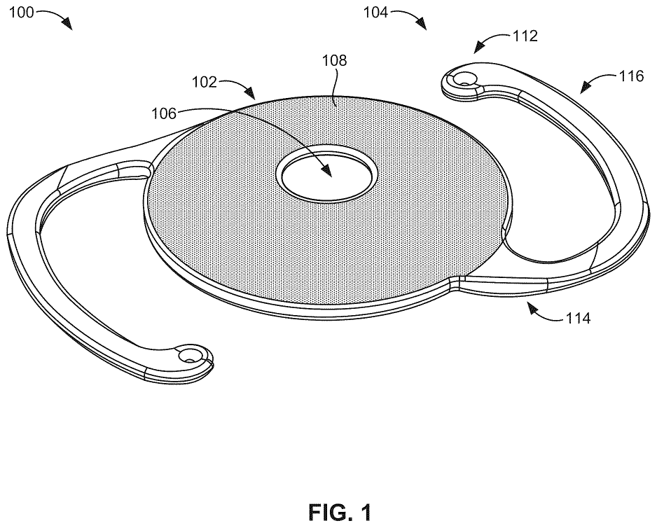

FIG. 1 illustrates a perspective view of one embodiment of an intraocular phakic implant 100. In some embodiments, the intraocular phakic implant 100 can comprise an opaque portion 102 and a plurality of haptics 104 coupled to or extending from the opaque portion 102.

The opaque portion 102 can comprise a pinhole opening 106 defined along the opaque portion 102. In some embodiments, the pinhole opening 106 can be substantially centered or located in a middle of the opaque portion 102. For example, the pinhole opening 106 can overlap with a midpoint of the opaque portion 102.

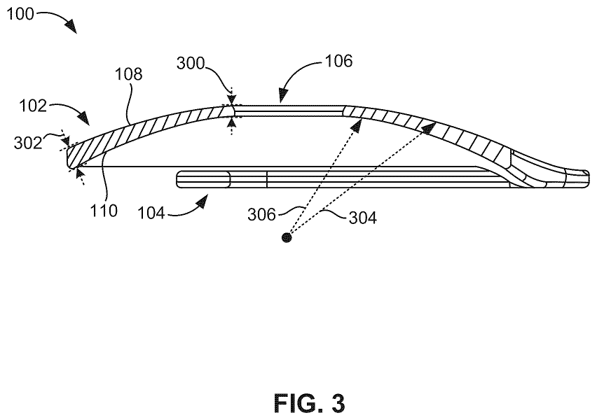

The opaque portion 102 can be shaped to allow at least part of the opaque portion 102 to be implanted or otherwise located between the natural crystalline lens of a subject and the iris of the subject. For example, in certain embodiments, the opaque portion 102 can be shaped substantially as a dome or flattened dome (see, e.g., FIG. 3).

Unlike pinhole-type intraocular lenses (IOLs), the opaque portion 102 is devoid of any refractive power or dioptric power. Moreover, the pinhole opening 106 is an actual (physical) opening or aperture rather than a transparent region of an otherwise opaque optic portion.

The intraocular phakic implant 100, including the opaque portion 102, can be made of a soft and flexible biocompatible polymeric material. For example, the intraocular phakic implant 100 can be made at least in part of a soft acrylic material.

In some embodiments, the entire intraocular phakic implant 100 can be made at least in part of a hydrophobic acrylic material. In certain embodiments, the opaque portion 102 of the intraocular phakic implant 100 can be made at least in part of a hydrophobic acrylic material. As a more specific example, the intraocular phakic implant 100 can be made at least in part of a hydrophobic acrylate/methacrylate copolymer.

In certain embodiments, the intraocular phakic implant 100 can be made at least in part of a hydrophobic acrylic material comprising hydroxyethyl methacrylate (HEMA) and/or hydroxyethyl acrylate (HEA).

In some embodiments, the intraocular phakic implant 100 can be made at least in part of a cross-linked copolymer comprising a copolymer blend. For example, the copolymer blend can comprise an alkyl acrylate or methacrylate, a fluoro-alkyl (meth)acrylate, and a phenyl-alkyl acrylate.

It is contemplated by this disclosure and it should be understood by one of ordinary skill in the art that these types of acrylic cross-linked copolymers can be generally copolymers of a plurality of acrylates, methacrylates, or a combination thereof and the term “acrylate” as used herein can be understood to mean acrylates, methacrylates, or a combination thereof interchangeably, unless otherwise specified. For example, the cross-linked copolymer can comprise an alkyl acrylate or methacrylate, a fluoro-alkyl acrylate or fluoro-alkyl methacrylate, and a phenyl-alkyl acrylate. In some embodiments, the cross-linked copolymer can comprise or be made in part of an n-butyl acrylate as the alkyl acrylate, trifluoroethyl methacrylate as the fluoro-alkyl acrylate, and phenylethyl acrylate as the phenyl-alkyl acrylate.

The final composition of the cross-linked copolymer can also comprise a cross-linker or cross-linking agent, such as ethylene glycol dimethacrylate (EGDMA), a hydroxyl-functional acrylic monomer (e.g., HEMA or HEA), certain initiators or initiating agents, and a UV absorber.

In other embodiments, the intraocular phakic implant 100 can be made at least in part of a hydrophilic acrylic material. In certain embodiments, the opaque portion 102 of the intraocular phakic implant 100 can be made at least in part of a hydrophilic acrylic material.

In further embodiments, the intraocular phakic implant 100 can be made at least in part of a biocompatible silicone. In certain embodiments, the opaque portion 102 of the intraocular phakic implant 100 can be made at least in part of a biocompatible silicone.

In additional embodiments, the intraocular phakic implant 100 can be made at least in part of polysiloxane. In certain embodiments, the opaque portion 102 of the intraocular phakic implant 100 can be made at least in part of polysiloxane.

In alternative embodiments, the intraocular phakic implant 100 can be made at least in part of poly(methyl methacrylate) (PMMA). In some embodiments, the opaque portion 102 of the intraocular phakic implant 100 can be made at least in part of PMMA.

In some embodiments, the opaque portion 102 can comprise an anterior exterior surface 108 and a posterior exterior surface 110 (see, e.g., FIG. 3). The anterior exterior surface 108 and the posterior exterior surface 110 can have a lubricious or slippery surface texture. The lubricious or slippery surface texture is to protect the iris and the crystalline lens since the opaque portion 102 of the intraocular phakic implant 100 will be implanted or located in between the iris and the crystalline lens.

As previously mentioned, the pinhole opening 106 can be a physical void or opening to allow fluid to flow through. For example, the pinhole opening 106 can allow aqueous humor and other fluid to flow through. The aqueous humor can carry nutrients from the posterior chamber to the anterior chamber of the eye where it is absorbed and eventually flows out to the periphery of the anterior chamber. The pinhole opening 106 can also allow outflow of any residual surgical fluids such as saline, nutrient-enriched saline, and/or viscoelastic fluids left behind during the surgery.

One technical problem faced by the applicant is how to design an intraocular phakic implant to be implanted in between the iris and the crystalline lens where the intraocular phakic implant would not hinder the flow of aqueous humor and other fluids left behind during the surgery. One technical solution discovered and developed by the applicant is the intraocular phakic implant 100 disclosed herein comprising a pinhole opening 106 defined along the opaque portion 102 of the intraocular phakic implant 100. The pinhole opening 106 is a physical aperture or hole that allows aqueous humor and other fluids left behind during the surgery to flow through.

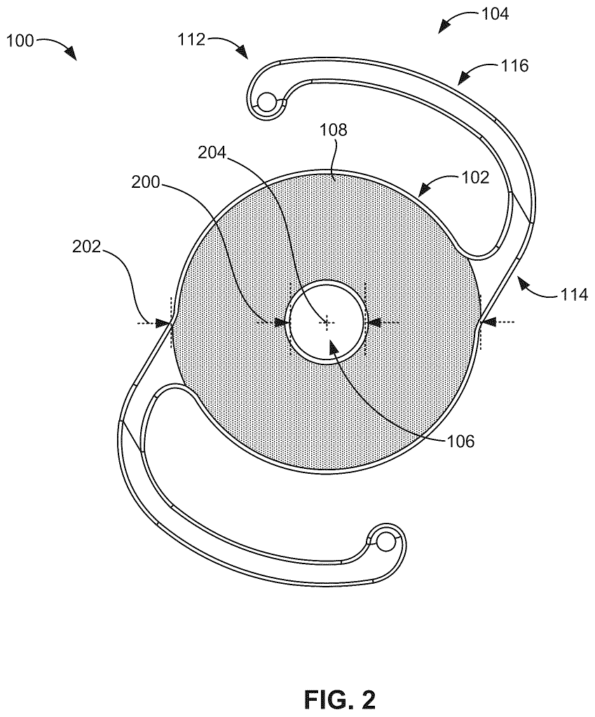

As shown in FIGS. 1 and 2, in some embodiments, the pinhole opening 106 can be substantially circular. In these embodiments, the pinhole opening 106 can have a pinhole diameter. The pinhole diameter can be between about 1.0 mm and about 3.0 mm.

In other embodiments, the pinhole opening 106 can be substantially elliptical-shaped or oval-shaped. In these embodiments, the pinhole opening 106 can have a maximum width. In certain embodiments, the maximum width of the pinhole opening 106 can be between about 1.0 mm and about 3.0 mm.

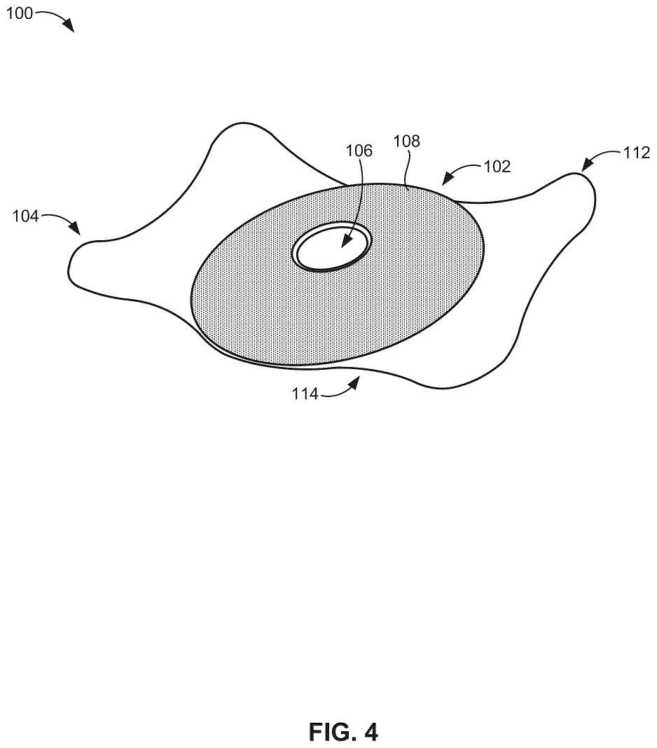

As shown in FIGS. 1 and 2, each of the haptics 104 can be an arm-type haptic shaped as a partial spiral or a barred spiral. In other embodiments, each of the haptics 104 can be a plate-type haptic or wing-type haptic (see, e.g., FIG. 4). The haptics 104 can stabilize and center the opaque portion 102 within the posterior chamber of the eye.

Each of the haptics 104 can comprise a haptic distal end 112, a haptic proximal end 114 coupled to the opaque portion 102, and a haptic intermediate segment 116 disposed in between the haptic proximal end 114 and the haptic distal end 112. As shown in FIG. 1, the haptic distal end 112 and the haptic intermediate segment 116 can be spaced apart from the opaque portion 102 and not connected to the opaque portion 102 when the haptic 104 is an arm-type haptic.

In some embodiments, the intraocular phakic implant 100 can be a one-piece implant such that the haptics 104 are connected to and extend from the opaque portion 102. In these embodiments, the haptics 104 are formed along with the opaque portion 102 and are not adhered or otherwise coupled to the opaque portion 102 in a subsequent step. In other embodiments, the haptics 104 can be coupled to and adhered to the opaque portion 102. For example, the haptics 104 can be adhered to the opaque portion 102 after each is formed separately.

In certain embodiments, each of the haptics 104 can be configured to bend or curl in an anterior direction to allow the haptics 104 to engage with the ciliary sulcus within the eye to fixate or otherwise secure the intraocular phakic implant 100 within the posterior chamber of the eye. For example, the haptic intermediate segment 116 and the haptic distal end 112 can be configured or otherwise formed to bend or curl in the anterior direction.

The haptics 104 can be configured to bend or curl in the anterior direction to prevent the haptics 104 from inadvertently damaging the zonules or interfering with the zonules. The zonules work with the ciliary muscles within the eye to help the crystalline lens undergo accommodation.

In some embodiments, the stiffness or rigidity of the haptics 104 can be greater than the stiffness or rigidity of the opaque portion 102. This can allow at least part of the haptics 104 to be bent, raised, or otherwise curled in the anterior direction when the intraocular phakic implant 100 is implanted within the posterior chamber of the eye.

In some embodiments, the opaque portion 102 can have rounded or smoothed-out edges and ends to prevent the opaque portion 102 from inadvertently damaging the capsular bag, the natural crystalline lens, and/or the zonules. In these and other embodiments, the haptics 104 can also have rounded or smoothed-out edges and ends to prevent the haptics 104 from damaging the capsular bag, the natural crystalline lens, and/or the zonules.

As previously discussed, the opaque portion 102 can be made of a biocompatible polymeric material. In some embodiments, the opaque portion 102 can be formed by directing laser light (e.g., from a femtosecond laser) at the biocompatible polymeric material until the biocompatible polymeric material carbonizes and turns opaque or black. For example, the laser light can have a wavelength of between about 1030 nm and 1064 nm (e.g., about 1053 nm). In certain embodiments, the laser light can have a pulse duration of between about 10,000 femtoseconds to about 10 femtoseconds and a pulse energy of between about 1 nanojoule (nJ) and 200 nJ.

In other embodiments, the biocompatible polymeric material making up the opaque portion 102 can comprise a black-colored pigment. For example, the opaque portion 102 can comprise the pigment carbon black. For example, the carbon black pigment can be distributed throughout the opaque portion 102 to cause this portion of the intraocular phakic implant 100 to appear black or opaque.

In some embodiments, the black-colored pigment (e.g., carbon black) can be added or mixed in as part of the manufacturing process for the intraocular phakic implant 100. In additional embodiments, the black-colored pigment can be part of a polymeric insert that is over-molded with a clear optical polymeric material (e.g., clear acrylic material) making up the other parts of the intraocular phakic implant 100. In further embodiments, the black-colored pigment can be printed on as one or more layers of the intraocular phakic implant 100.

FIG. 2 illustrates a top plan view or en face view of the intraocular phakic implant 100 of FIG. 1. As shown in FIG. 2, the pinhole opening 106 can have an opening diameter 200 when the pinhole opening 106 is substantially circular-shaped. In some embodiments, the opening diameter 200 can be between about 1.0 mm and about 3.0 mm. For example, the opening diameter 200 can be between about 1.5 mm and about 2.0 mm (e.g., about 1.8 mm).

The opaque portion 102 can also have a portion outer diameter 202. In some embodiments, the portion outer diameter 202 can be between about 6.0 mm and about 8.0 mm. For example, the portion outer diameter 202 can be between about 6.5 mm and about 7.5 mm.

In some embodiments, the intraocular phakic implant 100 can have a total device length as measured from one of the haptic distal ends 112 of a first haptic to another one of the haptic distal ends 112 of a second haptic (in instances where the intraocular phakic implant 100 has two haptics 104). For example, the total device length can be between about 10.0 mm and about 15.0 mm. As a more specific example, the total device length can be between about 11.0 mm and about 13.0 mm.

As shown in FIG. 2, the opaque portion 102 can be substantially shaped as an opaque annulus. In alternative embodiments, the pinhole opening 106 can be substantially shaped as an ellipse or oval.

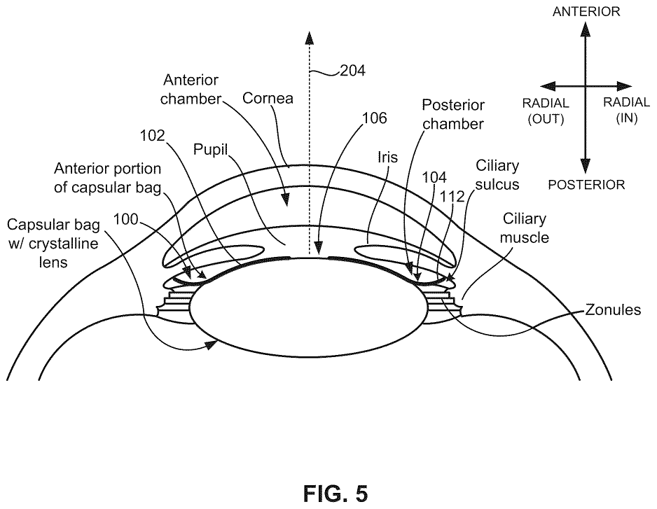

In certain embodiments, the intraocular phakic implant 100 can be positioned such that the pinhole opening 106 is aligned with or otherwise overlaps with a visual axis 204 of an eye of the subject when the intraocular phakic implant 100 is implanted within the eye (see, e.g., FIG. 5).

FIG. 3 illustrates a side cross-sectional view of part the intraocular phakic implant 100. The opaque portion 102 can be curved or substantially shaped as a dome or flattened dome when viewing a cross-section of the opaque portion 102 from the side. The dome-shape of the opaque portion 102 can allow the opaque portion 102 to conform to a shape of the natural crystalline lens of the subject (see, e.g., FIG. 5).

In some embodiments, the opaque portion 102 can have a thickness of between about 0.1 mm and about 0.5 mm. For example, the opaque portion 102 can have a minimum thickness 300 near a top or apex of the opaque portion 102 and a maximum thickness 302 near a periphery of the opaque portion 102. In some embodiments, the minimum thickness 300 can be between about 0.1 mm and 0.2 mm and the maximum thickness 302 can be between about 0.3 mm and 0.5 mm.

FIG. 3 also illustrates that the anterior exterior surface 108 of the opaque portion 102 can have an anterior radius of curvature 304 and the posterior exterior surface 110 can have a posterior radius of curvature 306. In some embodiments, the anterior radius of curvature 304 can be between about 7.0 mm and 9.0 mm (e.g., about 8.0 mm). In these and other embodiments, the posterior radius of curvature 306 can be between about 5.0 mm and 7.0 mm (e.g., about 6.0 mm).

Although FIG. 3 illustrates the intraocular phakic implant 100 comprising an arm-type haptic 104, it is contemplated by this disclosure and it should be understood by one of ordinary skill in the art that the shape and dimensions of the opaque portion 102 depicted in FIG. 3 can also apply to opaque portions 102 of intraocular phakic implants 100 comprising plate-type or wing-type haptics (see, e.g., FIG. 4).

FIG. 4 illustrates a perspective view of another embodiment of an intraocular phakic implant 100 comprising haptics 104 shaped or configured as plates or wings. Similar to the intraocular phakic implant 100 depicted in FIG. 1, this embodiment of the intraocular phakic implant 100 can also comprise a curved or dome-shaped opaque portion 102 and a plurality of haptics 104 coupled to or extending from the opaque portion 102.

The opaque portion 102 can comprise a pinhole opening 106 substantially centered or located in a middle of the opaque portion 102. The opaque portion 102 can be shaped to allow at least part of the opaque portion 102 to be implanted, positioned, or otherwise located in between the natural crystalline lens of a subject and the iris of the subject (see, e.g., FIG. 5).

The opaque portion 102 is devoid of any refractive power or dioptric power. Moreover, the pinhole opening 106 is an actual (physical) opening or aperture rather than a transparent region of an otherwise opaque optic portion.

The opaque portion 102 can be made of a soft and flexible biocompatible polymeric material. For example, the opaque portion 102 can be made at least in part of a soft acrylic material.

In some embodiments, the opaque portion 102 can be made at least in part of a hydrophobic acrylic material, a hydrophilic acrylic material, silicone, polysiloxane, or poly(methyl methacrylate) (PMMA).

In some embodiments, the stiffness or rigidity of the plate-type or wing-type haptics 104 can be greater than the stiffness or rigidity of the opaque portion 102. This can allow at least part of the haptics 104 to be bent, raised, flared-up, or otherwise set in the anterior direction.

As shown in FIG. 4, the haptic distal ends 112 of the plate-type or wing-type haptics can be bent, raised, flared-up, or otherwise set in an anterior direction. This can ensure that the haptic distal ends 112 engage with the ciliary sulcus to fixate or secure the intraocular phakic implant 100 within the posterior chamber of the eye and do not inadvertently damage or interfere with the zonules.

As shown in FIG. 4, the opaque portion 102 and the haptics 104 can have rounded or smoothed-out edges and ends to prevent the opaque portion 102 and the haptics 104 from inadvertently damaging the capsular bag, the natural crystalline lens, and/or the zonules.

One technical problem faced by the applicant is how to design the intraocular phakic implant 100 such that the haptics 104 of the intraocular phakic implant 100 does not inadvertently damage the zonules or the crystalline lens. One technical solution discovered and developed by the applicant are the haptics 104 disclosed herein where the haptic distal ends 112 are bent or raised to allow the haptics 104 to engage with the ciliary sulcus when the intraocular phakic implant 100 is implanted in between the iris and the crystalline lens in the posterior chamber of the eye.

FIG. 5 is a schematic diagram illustrating a side cross-sectional view of the intraocular phakic implant 100 implanted within the eye. As shown in FIG. 5, the intraocular phakic implant 100 is implanted within the posterior chamber of the eye behind the iris. For example, the intraocular phakic implant 100 is implanted within the posterior chamber of the eye in between the iris and the capsular bag containing the natural crystalline lens of the subject.

In some embodiments, the intraocular phakic implant 100 is implanted within a non-dominant eye of the subject.

The opaque portion 102 can conform to a curvature of the crystalline lens of the subject. As shown in FIG. 5, the haptic distal ends 112 of the haptics 104 can be bent or curled in an anterior direction to allow the haptics 104 to engage with the ciliary sulcus to fixate or secure the intraocular phakic implant 100 within the posterior chamber of the eye. This can prevent the haptic distal ends 112 from inadvertently damaging or interfering with the zonules.

When implanted within the eye of the subject, the intraocular phakic implant 100 can act as an artificial iris and limit the aperture of the eye and create a pinhole effect to increase a depth of focus of the eye.

In some embodiments, the intraocular phakic implant 100 can be implanted within a non-dominant eye of a subject. In these embodiments, the intraocular phakic implant 100 can act as an artificial iris of the non-dominant eye and increase the depth of focus of the non-dominant eye.

In certain embodiments, the intraocular phakic implant 100 can be implanted within an eye to treat a highly aberrated cornea or high-order aberrations of the cornea. For example, a method of treating a highly aberrated cornea or high-order aberrations of the cornea can comprise forming an incision in a cornea of an eye in operation and introducing the intraocular phakic implant 100 into a posterior chamber of the eye behind the iris of the eye.

Moreover, as shown in FIG. 5, the intraocular phakic implant 100 can be positioned such that the pinhole opening 106 overlaps with the visual axis 204 of the eye. In some embodiments, the visual axis 204 of the eye can be approximated to be halfway between the pupil center and the corneal vertex.

In other embodiments, the visual axis 204 of the eye can be determined by a process disclosed in U.S. Pat. Pub. No. 2021/0369105, the content of which is incorporated herein by reference in its entirety.

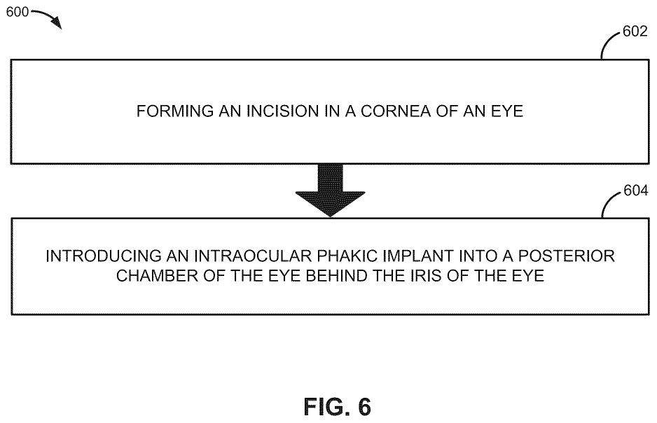

FIG. 6 illustrates one embodiment of a method 600 of correcting for presbyopia with an intraocular phakic implant. The method 600 can comprise forming an incision in a cornea of an eye in operation 602. In some embodiments, operation 602 can comprise forming an incision sized between about 1.5 mm and 2.0 mm (e.g., about 1.8 mm) in the cornea of the eye. For example, the eye can be the non-dominant eye of the subject.

The method 600 can further comprise introducing the intraocular phakic implant 100 into a posterior chamber of the eye behind the iris of the eye in operation 604. In some embodiments, operation 604 can further comprise introducing the intraocular phakic implant 100 into the posterior chamber of the eye until the intraocular phakic implant 100 is implanted or positioned between the crystalline lens of the eye and the iris of eye.

In some embodiments, the method 600 can further comprise rolling up the intraocular phakic implant 100 and introducing the intraocular phakic implant 100 into the eye when the intraocular phakic implant is rolled-up or in a rolled-up configuration.

In certain embodiments, the method 600 can further comprise removing the intraocular phakic implant 100 from the eye after a period of time. More specifically, the method 600 can further comprise removing the intraocular phakic implant 100 from the eye prior to pseudophakic cataract surgery.

One technical problem faced by the applicant is how to provide a safe and effective treatment for emerging presbyopic patients (e.g., those between the ages of 35 and 45). These patients may not be ready for cataract surgery but may benefit from an implant that addresses their emerging or early presbyopia. One technical solution discovered and developed by the applicant is the method disclosed herein involving introducing the intraocular phakic implant 100 into a posterior chamber of the eye (e.g., a non-dominant eye) of the subject behind the iris of the eye. The intraocular phakic implant 100 can act like an artificial iris and increase the depth of focus of the eye (e.g., the non-dominant eye) via the pinhole effect. The method can also comprise removing the intraocular phakic implant from the eye (e.g., the non-dominant eye) prior to the subject undergoing cataract surgery.

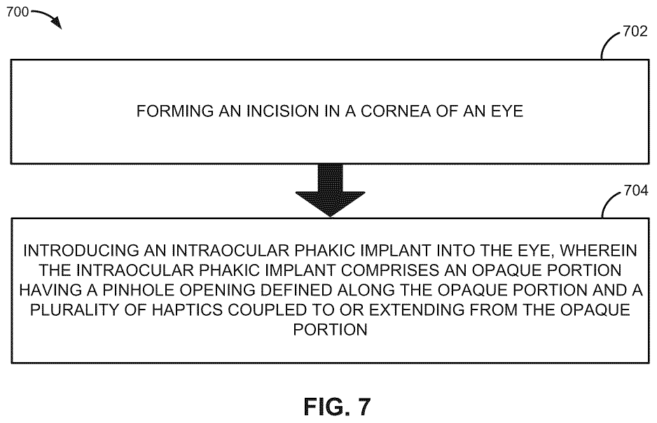

FIG. 7 illustrates one embodiment of a method 700 for increasing a depth of focus of an eye. The method 700 can comprise forming an incision in a cornea of an eye in operation 702. In some embodiments, operation 702 can comprise forming an incision sized between about 1.5 mm and 2.0 mm (e.g., about 1.8 mm) in the cornea of the eye. For example, the eye can be the non-dominant eye of the subject.

The method 700 can further comprise introducing the intraocular phakic implant 100 into the eye in operation 704. The intraocular phakic implant 100 can comprise the opaque portion 102 having a pinhole opening 106 defined along the opaque portion 102 and a plurality of haptics 104 coupled to or extending from the opaque portion 102.

In some embodiments, the operation 704 can further comprise introducing the intraocular phakic implant 100 into the posterior chamber of the eye until the intraocular phakic implant 100 is implanted or positioned between the crystalline lens of the eye and the iris of eye.

One technical problem faced by the applicant is where to implant the intraocular phakic implant 100 disclosed herein such that the implant is not visible when implanted within the eye of the subject. For example, cornea inlays are another type of device for correcting presbyopia. However, cornea inlays are visible when implanted within the eye and can appear unsightly. One technical solution discovered and developed by the applicant is to implant the intraocular phakic implant 100 disclosed herein behind the iris and, more specifically, in between the iris and the crystalline lens of the subject. This also allows the intraocular phakic implant 100 to be easily removed when it comes time for the subject to undergo cataract surgery.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of” when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having,” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially,” “about,” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An intraocular phakic implant, comprising:
an opaque portion comprising a pinhole opening defined along the opaque portion; and
a plurality of haptics coupled to or extending from the opaque portion.

2. The intraocular phakic implant of claim 1, wherein the opaque portion is devoid of refractive power.

3. The intraocular phakic implant of claim 1, wherein each of the haptics is an arm-type haptic shaped as a partial spiral.

4. The intraocular phakic implant of claim 1, wherein each of the haptics is a plate-type haptic.

5. The intraocular phakic implant of claim 1, wherein each of the haptics is configured to bend or curl in an anterior direction to allow the haptics to engage with the ciliary sulcus to fixate the intraocular phakic implant within an eye.

6. The intraocular phakic implant of claim 1, wherein a stiffness or rigidity of the haptics is greater than the stiffness or rigidity of the opaque portion.

7. The intraocular phakic implant of claim 1, wherein the opaque portion comprises a lubricious surface texture.

8. The intraocular phakic implant of claim 1, wherein the opaque portion is shaped substantially as a flattened dome.

9. The intraocular phakic implant of claim 1, wherein the opaque portion has a thickness of between about 0.1 mm and about 0.5 mm.

10. The intraocular phakic implant of claim 1, wherein the pinhole opening has an opening diameter, wherein the opening diameter is between about 1.0 mm and about 3.0 mm.

11. The intraocular phakic implant of claim 1, wherein the opaque portion has a portion outer diameter, wherein the portion outer diameter is between about 6.0 mm and about 8.0 mm.

12. The intraocular phakic implant of claim 1, wherein the pinhole opening is substantially circular.

13. The intraocular phakic implant of claim 1, wherein the pinhole opening is configured to allow aqueous humor and other fluids to flow through the pinhole opening.

14. The intraocular phakic implant of claim 1, wherein the opaque portion is made of a biocompatible polymeric material, and wherein the opaque portion is formed by directing laser light at the biocompatible polymeric material until the biocompatible polymeric material carbonizes and turns opaque.

15. The intraocular phakic implant of claim 1, wherein the opaque portion comprises a black-colored pigment.

16. The intraocular phakic implant of claim 1, wherein the opaque portion and the haptics comprise rounded edges and ends.

17. A method of correcting for presbyopia, comprising:
forming an incision in a cornea of an eye; and
introducing an intraocular phakic implant into a posterior chamber of the eye until the intraocular phakic implant is implanted or positioned between the crystalline lens of the eye and the iris of the eye.

18. The method of claim 17, wherein the intraocular phakic implant comprises an opaque portion comprising a pinhole opening defined along the opaque portion.

19. The method of claim 18, wherein the pinhole opening is configured to allow aqueous humor and other fluids within the eye to flow through the pinhole opening.

20-33. (canceled)

34. A method of increasing a depth of focus of an eye, comprising:
forming an incision in a cornea of the eye; and
introducing an intraocular phakic implant into a posterior chamber of the eye, wherein the intraocular phakic implant comprises:
an opaque portion comprising a pinhole opening defined along the opaque portion, and
a plurality of haptics coupled to or extending from the opaque portion.

35-50. (canceled)
